(19) United States
(12) Patent Application Publication
(10) Pub. No.: US 2025/0262218 A1
(43) Pub. Date: Aug. 21, 2025

(54) COMPOSITIONS, FORMULATIONS AND METHODS FOR STIMULATING RESPIRATION

(71) Applicant: Auros Pharma LLC, Johnson City, TN (US)

(72) Inventor: Joseph Michael NOUNOU, Johnson City, TN (US)

(21) Appl. No.: 18/637,263

(22) Filed: Apr. 16, 2024

Related U.S. Application Data
(60) Provisional application No. 63/555,718, filed on Feb. 20, 2024.

(51) Int. Cl. A61K 31/5377; A61K 9/00; A61K 9/107; A61K 31/485

(52) U.S. Cl. CPC A61K 31/5377; A61K 9/0019; A61K 9/107; A61K 31/485

(57) ABSTRACT

Disclosed herein are devices, compositions, and methods for stimulating respiration using a respiratory stimulant and an opioid antagonist.

### CROSS-REFERENCE TO RELATED APPLICATIONS

This application claims priority to U.S. Application No. 63/555,718, filed on Feb. 20, 2024, which is hereby incorporated by reference in its entireties for all purposes.

### BACKGROUND OF THE INVENTION

Drug overdose is a major social issue that affects all aspects of society. Drug overdose in hospitals caused by opioids (such as heroin, fentanyl, oxycodone, hydrocodone, codeine, and morphine) can often be treated using naloxone, which is an opioid antagonist. Naloxone can quickly restore normal breathing to a person if their breathing has slowed or stopped because of an opioid overdose. However, naloxone has little effect on high potency synthetic opiates like IMF (illegally manufactured fentanyl) seen in the community and non-opioid drug overdose, for example, caused by a stimulant such as methamphetamine or benzodiazepines.

There remains a need for methods and compositions for the treatment drug overdose caused by opioid, a non-opioid drug, a combination of both, as well as other respiratory arrest conditions at home, where immediate access to a ventilator may not be available. For example, the compositions and methods here may be used for patients with respiratory arrest due to cardiopulmonary arrest or chronic obstructive pulmonary disease (COPD) exacerbations, potentially providing temporary respiratory support until respiration is recovered in a hospital setting.

### SUMMARY OF THE INVENTION

Provided herein are pharmaceutical compositions comprising doxapram. In aspects, the pharmaceutical composition comprises: i) a unit dose of doxapram from about 5 mg to about 600 mg; and ii) a lipid emulsion. In aspects, the unit dose of doxapram is about 50 to about 150 mg. In aspects, the lipid emulsion comprises soy bean oil, egg phospholipids, glycerin, or any combination thereof. In aspects, the lipid emulsion ranges from about 0.1% to about 30% (wt) of the pharmaceutical composition. In aspects, the doxapram is encapsulated in the lipid emulsion as a sustained release dosage form of doxapram. In aspects, the doxapram is about 3 mg/mL. In aspects, the lipid emulsion is at a concentration of about 1.5% (wt) of the pharmaceutical composition, wherein the lipid emulsion comprises 20% soybean oil, 1.2% egg phospholipids, 2.25% glycerin, and water, and wherein the doxapram is either in racemate or (S)-enantiomer form.

Provided herein are devices. In aspects, the device comprises at least one container, the at least one container comprising a pharmaceutical composition comprising a sustained release dosage form of a respiratory stimulant and a lipid emulsion. In aspects, the at least one container comprises a pharmaceutical composition disclosed herein. In aspects, the device comprises i) a first container comprising a first pharmaceutical composition, wherein the first pharmaceutical composition comprises the respiratory stimulant and an opioid antagonist; and ii) a second container comprising a second pharmaceutical composition, wherein the second pharmaceutical composition comprises the sustained release dosage form of the respiratory stimulant and the lipid emulsion. In aspects, the first pharmaceutical composition comprises a unit dose of the opioid antagonist of about 0.05 mg to about 10 mg and a unit dose of the respiratory stimulant of about 5 mg to about 300 mg and the second pharmaceutical composition comprises a unit dose of the respiratory stimulant of about 10 mg to about 600 mg. In aspects, the first pharmaceutical composition comprises a unit dose of the opioid antagonist of about 1 mg to about 3 mg and a unit dose of the respiratory stimulant of about 50 mg to about 150 mg and the second pharmaceutical composition comprises a unit dose of the respiratory stimulant of about 100 mg to about 300 mg. In aspects, the first pharmaceutical composition comprises a unit dose of the opioid antagonist of about 0.01 mg/kg body weight to about 1 mg/kg body weight and a unit dose of the respiratory stimulant of about 0.1 mg/kg body weight to about 10 mg/kg body weight and the second pharmaceutical composition comprises a unit dose of the respiratory stimulant of about 0.2 mg/kg body weight to about 20 mg/kg body weight. In aspects, the lipid emulsion encapsulates the respiratory stimulant, and wherein the lipid emulsion ranges from about 0.1% to about 90% (wt) of the second pharmaceutical composition. In aspects, the lipid emulsion is about 0.1% to about 30% (wt) of the second pharmaceutical composition. In aspects, the lipid emulsion comprises soybean oil, egg phospholipids, glycerin, or any combination thereof. In aspects, the respiratory stimulant comprises doxapram, nikethamide, pentetrazol, etamivan, bemegride, prethcamide, almitrine, dimefline, or mepixanox and wherein the opioid antagonist comprises naloxone, naltrexone, nalbuphine, butorphanol, pentazocine, diprenorphine, or dihydroetorphine. In aspects, the first pharmaceutical composition comprises doxapram and naloxone, and the second pharmaceutical composition comprises the sustained release dosage form of doxapram.

Provided herein are devices comprising: i) a first container comprising a first pharmaceutical composition, wherein the first pharmaceutical composition comprises doxapram and naloxone; and ii) a second container comprising a second pharmaceutical composition, wherein the second pharmaceutical composition comprises a sustained release dosage form of doxapram and a lipid emulsion, wherein the first pharmaceutical composition comprises a unit dose of naloxone of about 1 mg to about 3 mg and a unit dose of doxapram of about 50 mg to about 150 mg and the second pharmaceutical composition comprises a unit dose of doxapram of about 100 mg to about 300 mg, and wherein the lipid emulsion is about 0.1% to about 30% (wt) of the second pharmaceutical composition. In aspects, the device is suitable for administering the first and second pharmaceutical compositions. In aspects, the first and second pharmaceutical compositions are administered using an auto-injector, wherein the administration occurs sequentially or concurrently.

Provided herein are methods. In aspects, a method comprises stimulating respiration, comprising administering a pharmaceutical composition to a subject in need thereof, wherein the pharmaceutical composition comprises: i) a unit dose of doxapram from about 5 mg to about 600 mg; and ii) a lipid emulsion. In aspects, the lipid emulsion comprises soy bean oil, egg phospholipids, glycerin, or any combination thereof. In aspects, the lipid emulsion ranges from about 0.1% to about 30% (wt) of the pharmaceutical composition. In aspects, the lipid emulsion ranges from about 0.5% to about 1.5% (wt) of the pharmaceutical composition. In aspects, the subject has drug-induced post-anesthesia respiratory depression, apnea, respiratory or central nervous system depression due to drug overdose, acute respiratory insufficiency superimposed on chronic obstructive pulmonary disease (COPD), or cardiopulmonary arrest. In aspects, a method comprises a first pharmaceutical composition and a second first pharmaceutical composition, wherein the first pharmaceutical composition comprises a unit dose of naloxone of about 1 mg to about 3 mg and a unit dose of doxapram of about 50 mg to about 150 mg and the second pharmaceutical composition comprises a unit dose of doxapram of about 100 mg to about 300 mg, and wherein the lipid emulsion is about 0.1% to about 30% (wt) of the second pharmaceutical composition. In aspects, the subject has respiratory or central nervous system depression due to drug overdose. In aspects, the first pharmaceutical composition comprises a unit dose of naloxone of about 0.01 mg/kg body weight to about 1 mg/kg body weight and a unit dose of doxapram of about 0.1 mg/kg body weight to about 10 mg/kg body weight and the second pharmaceutical composition comprises a unit dose of doxapram of about 0.2 mg/kg body weight to about 20 mg/kg body weight. In aspects, the doxapram reaches a maximum plasma concentration (Cmax) in about 15 minutes to about 30 minutes and maintains a plasma concentration of more than about 5% of said Cmax for at least once hour, after a single dose of said pharmaceutical composition by intramuscular injection.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary device (e.g., auto-injector) described herein.

FIG. 2 shows exemplary pharmacokinetics of doxapram in various concentrations of lipid emulsion (0%, 0.5%, and 1.5%) administered intramuscularly to canines. Blood samples were taken at baseline (0 minutes) and every 5 minutes over the course of 60 minutes to measure concentration (ng/dL) of doxapram. The same three canines (beagles) were assessed at all lipid emulsion concentrations and doxapram concentrations were averaged for each study arm. Lipid emulsion formulation: 20% soybean oil, 1.2% egg phospholipids, 2.25% glycerin, and water.

### DETAILED DESCRIPTION OF THE INVENTION

### Definitions

The singular forms “a”, “an”, and “the” are used herein to include plural references unless the context clearly dictates otherwise. Accordingly, unless the contrary is indicated, the numerical parameters set forth in this application are approximations that may vary depending upon the desired properties sought to be obtained by the present invention.

The term “about” and its grammatical equivalents in relation to a reference numerical value and its grammatical equivalents as used herein can include a range of values plus or minus 10% from that value, such as a range of values plus or minus 10%, 9%, 8%, 7%, 6%, 5%, 4%, 3%, 2%, or 1% from that value. For example, the amount “about 10” includes amounts from 9 to 11.

The term “dosage form” refers to a drug formulation which can be prescribed and/or administered to a subject and is meant to include any solid, semi-solid, liquid, suspension, frozen or freeze-dried preparation of a drug, including but not limited to, tablets, minitablets, soft or hard capsules, gel capsules, caplets, granules, pellets, micropellets, beads, powders, sachets, liquids, suspensions and the like.

The term “immediate release” or “IR” refers to dosage forms that are formulated to release an active drug immediately after administration (e.g., oral or parenteral); no deliberate effort is made to modify the drug release rate in immediate release dosage forms or formulations. Immediate-release products generally result in relatively rapid drug absorption and onset of accompanying pharmacodynamic effects. For example, immediate release dosage forms comprising a therapeutic agent can release at least about 50% of the therapeutic agent within about 5 minutes after the administration.

The term “lipid emulsion” refers to an emulsion of lipid for pharmaceutical use (e.g., for human or veterinary use, such as canine). For example, lipid emulsion can refer to a commercially available version sold under the trademark INTRALIPID™, which is an emulsion of soy bean oil, egg phospholipids and glycerin, and is available in 10%, 20% and 30% concentrations.

The term “opioid antagonist” is a drug which acts to block one or more of the opioid receptors in the central or peripheral nervous system, such as naloxone, naltrexone, nalbuphine, butorphanol, pentazocine, diprenorphine, and dihydroetorphine.

The term “pharmaceutically acceptable salt” is intended to mean those salts that retain one or more of the biological activities and properties of the free acids and bases and that are not biologically or otherwise undesirable. Illustrative examples of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycolates, tartrates, methanesulfonates, propanesulfonates, naphthalene-1-sulfonates, naphthalene-2-sulfonates, and mandelates.

Unless otherwise indicated, the chemical compound names disclosed herein include any pharmaceutically acceptable salt thereof. For example, the term “doxapram” refers to the chemical compound 1-ethyl-4-(2-morpholinoethyl)-3,3-diphenyl-2-pyrrolidinone (CAS number 309-29-5), as well as its pharmaceutically acceptable salt, e.g., doxapram hydrochloride (1-ethyl-4-(2-morpholinoethyl)-3,3-diphenyl-2-pyrrolidinone monohydrochloride, monohydrate). Similarly, the term “naloxone” also includes its pharmaceutically acceptable salt, e.g., naloxone hydrochloride.

The term “respiratory stimulant” is a drug which acts to increase the action of the respiratory system, such as doxapram, nikethamide, pentetrazol, etamivan, bemegride, prethcamide, almitrine, dimefline, and mepixanox.

The term “sustained release” or “SR” refers to a dosage form that (in contrast to immediate-release dosage forms) delivers a drug for a prolonged period of time; it is meant to include any dosage form or formulation which is not an immediate release dosage form or formulation including those described in Chapter 17 of “Applied Biopharmaceutics and Pharmacokinetics”, Sixth Edition; Shargel et al., which is incorporated herein by reference; thus for the purposes herein, “sustained release” or “SR” dosage forms can be used interchangeably with “extended release” or “ER” dosage forms, or “delayed release” or “DR” dosage forms. For example, a sustained release dosage form can release less than about 50% of a therapeutic agent within about 5 minutes after the administration. In some cases, a sustained release dosage form can release more than about 50% of a therapeutic agent in a time frame from about 5 minutes to about 30 minutes after the administration.

Unless otherwise indicated, open terms for example “contain,” “containing,” “include,” “including,” and the like mean comprising.

Unless otherwise indicated, some embodiments herein contemplate numerical ranges. When a numerical range is provided, unless otherwise indicated, the range includes the range endpoints. Unless otherwise indicated, numerical ranges include all values and sub ranges therein as if explicitly written out.

### Compositions and Formulations

Disclosed herein is a first pharmaceutical composition comprising a respiratory stimulant and an opioid antagonist. In aspects, a respiratory stimulant can increase the action of the respiratory system. Exemplary respiratory stimulants include, but are not limited to, doxapram, nikethamide, pentetrazol, etamivan, bemegride, prethcamide, almitrine, dimefline, and mepixanox. In aspects, a opioid antagonist can block one or more of the opioid receptors in the central or peripheral nervous system. Exemplary opioid antagonists include, but are not limited to, naloxone, naltrexone, nalbuphine, butorphanol, pentazocine, diprenorphine, and dihydroetorphine.

In aspects, a respiratory stimulant comprises doxapram. In aspects, doxapram stimulates an increase in tidal volume and respiratory rate. In aspects, doxapram is a white to off-white, odorless, crystalline powder that is stable in light and air. In aspects, doxapram is soluble in water, sparingly soluble in alcohol and practically insoluble in ether. In aspects, benzyl alcohol or chlorobutanol is added as a preservative agent in a commercially available form of doxapram. In aspects, a commercially available form of doxapram has a pH from 3.5-5. In aspects, a commercially available form of doxapram is in injection form. Doxapram hydrochloride is sold under the trademark DOPRAM™. Doxapram can stimulate chemoreceptors in the carotid bodies of the carotid arteries, which in turn, stimulates the respiratory center in the brain stem. Doxapram can be used in intensive care settings to stimulate the respiratory rate in patients with respiratory failure. Doxapram can be useful for treating respiratory depression in patients who have taken excessive doses of drugs such as buprenorphine or fentanyl analogues. Doxapram can be as effective as pethidine in suppressing shivering after surgery.

In aspects, a unit dose of a respiratory stimulant (e.g., doxapram) is about 5 mg to about 300 mg. In aspects, a unit dose of a respiratory stimulant (e.g., doxapram) is at least about 5 mg. In aspects, a unit dose of a respiratory stimulant (e.g., doxapram) is at most about 300 mg. In aspects, a unit dose of a respiratory stimulant (e.g., doxapram) is about 5 mg to about 10 mg, about 5 mg to about 50 mg, about 5 mg to about 100 mg, about 5 mg to about 150 mg, about 5 mg to about 200 mg, about 5 mg to about 250 mg, about 5 mg to about 300 mg, about 10 mg to about 50 mg, about 10 mg to about 100 mg, about 10 mg to about 150 mg, about 10 mg to about 200 mg, about 10 mg to about 250 mg, about 10 mg to about 300 mg, about 50 mg to about 100 mg, about 50 mg to about 150 mg, about 50 mg to about 200 mg, about 50 mg to about 250 mg, about 50 mg to about 300 mg, about 100 mg to about 150 mg, about 100 mg to about 200 mg, about 100 mg to about 250 mg, about 100 mg to about 300 mg, about 150 mg to about 200 mg, about 150 mg to about 250 mg, about 150 mg to about 300 mg, about 200 mg to about 250 mg, about 200 mg to about 300 mg, or about 250 mg to about 300 mg. In aspects, a unit dose of a respiratory stimulant (e.g., doxapram) is about 5 mg, about 10 mg, about 50 mg, about 100 mg, about 150 mg, about 200 mg, about 250 mg, or about 300 mg. In aspects, a unit dose of a respiratory stimulant (e.g., doxapram) is about 50 mg to about 150 mg.

In aspects, a unit dose of a respiratory stimulant (e.g., doxapram) is about 0.1 mg/kg body weight to about 10 mg/kg body weight. In aspects, a unit dose of a respiratory stimulant (e.g., doxapram) is at least about 0.1 mg/kg body weight. In aspects, a unit dose of a respiratory stimulant (e.g., doxapram) is at most about 10 mg/kg body weight. In aspects, a unit dose of a respiratory stimulant (e.g., doxapram) is about 0.1 mg/kg body weight to about 0.2 mg/kg body weight, about 0.1 mg/kg body weight to about 0.5 mg/kg body weight, about 0.1 mg/kg body weight to about 1 mg/kg body weight, about 0.1 mg/kg body weight to about 2 mg/kg body weight, about 0.1 mg/kg body weight to about 5 mg/kg body weight, about 0.1 mg/kg body weight to about 10 mg/kg body weight, about 0.2 mg/kg body weight to about 0.5 mg/kg body weight, about 0.2 mg/kg body weight to about 1 mg/kg body weight, about 0.2 mg/kg body weight to about 2 mg/kg body weight, about 0.2 mg/kg body weight to about 5 mg/kg body weight, about 0.2 mg/kg body weight to about 10 mg/kg body weight, about 0.5 mg/kg body weight to about 1 mg/kg body weight, about 0.5 mg/kg body weight to about 2 mg/kg body weight, about 0.5 mg/kg body weight to about 5 mg/kg body weight, about 0.5 mg/kg body weight to about 10 mg/kg body weight, about 1 mg/kg body weight to about 2 mg/kg body weight, about 1 mg/kg body weight to about 5 mg/kg body weight, about 1 mg/kg body weight to about 10 mg/kg body weight, about 2 mg/kg body weight to about 5 mg/kg body weight, about 2 mg/kg body weight to about 10 mg/kg body weight, or about 5 mg/kg body weight to about 10 mg/kg body weight. In aspects, a unit dose of a respiratory stimulant (e.g., doxapram) is about 0.1 mg/kg body weight, about 0.2 mg/kg body weight, about 0.5 mg/kg body weight, about 1 mg/kg body weight, about 2 mg/kg body weight, about 5 mg/kg body weight, or about 10 mg/kg body weight.

In aspects, an opioid antagonist comprises naloxone. Naloxone, in some cases sold under the trademark NARCAN™, is a medication used to block the effects of opioids. Naloxone (CAS number 465-65-6), also known as N-allylnoroxymorphone or as 17-allyl-4,5α-epoxy-3,14-dihydroxymorphinan-6-one, is a synthetic morphinan derivative and was derived from oxymorphone (14-hydroxydihydromorphinone), an opioid analgesic. Oxymorphone, in turn, was derived from morphine, an opioid analgesic and naturally occurring constituent of the opium poppy. Naloxone is a racemic mixture of two enantiomers, (−)-naloxone (levonaloxone, i.e., S-enantiomer) and (+)-naloxone (dextronaloxone, i.e., R-enantiomer), only the former of which is active at opioid receptors. In aspects, naloxone is used in the racemate form. In aspects, naloxone is used in the (−)-naloxone (levonaloxone) form. Naloxone is highly lipophilic, allowing it to rapidly penetrate the brain and to achieve a far greater brain to serum ratio than that of morphine. Opioid antagonists related to naloxone include cyprodime, nalmefene, nalodeine, naloxol, and naltrexone.

Naloxone is often used to counter decreased breathing in opioid overdose. Naloxone can also be combined with an opioid (e.g., in the same pill), to decrease the risk of opioid misuse. When given intravenously, effects begin within two minutes, and when injected into a muscle within five minutes. Another route it can be given is by spraying it into a person's nose. In aspects, the effects of naloxone can last from about 30 seconds to 24 hours.

Naloxone can be injected intravenously, with an onset of 1-2 minutes and a duration of up to 45 minutes. While the onset is achieved fastest through intravenous administration (IV) than through other routes of administration, it may be difficult to obtain venous access in patients who use IV drugs chronically. This may be an issue under emergency conditions. Naloxone can also be administered via intramuscular or subcutaneous injection. The onset of naloxone provided through this route can be 2 to 5 minutes with a duration of around 30-120 min. Naloxone administered intramuscularly can be provided through pre-filled syringes, vials, and auto-injector (such as ZIMHI™). Naloxone can also be administered intranasally, for example, for people who are unconscious or unresponsive.

In aspects, a unit dose of an opioid antagonist (e.g., naloxone) is about 0.05 mg to about 10 mg. In aspects, a unit dose of an opioid antagonist (e.g., naloxone) is at least about 0.05 mg. In aspects, a unit dose of an opioid antagonist (e.g., naloxone) is at most about 10 mg. In aspects, a unit dose of an opioid antagonist (e.g., naloxone) is about 0.05 mg to about 0.1 mg, about 0.05 mg to about 0.5 mg, about 0.05 mg to about 1 mg, about 0.05 mg to about 3 mg, about 0.05 mg to about 5 mg, about 0.05 mg to about 10 mg, about 0.1 mg to about 0.5 mg, about 0.1 mg to about 1 mg, about 0.1 mg to about 3 mg, about 0.1 mg to about 5 mg, about 0.1 mg to about 10 mg, about 0.5 mg to about 1 mg, about 0.5 mg to about 3 mg, about 0.5 mg to about 5 mg, about 0.5 mg to about 10 mg, about 1 mg to about 3 mg, about 1 mg to about 5 mg, about 1 mg to about 10 mg, about 3 mg to about 5 mg, about 3 mg to about 10 mg, or about 5 mg to about 10 mg. In aspects, a unit dose of an opioid antagonist (e.g., naloxone) can be about 0.05 mg, about 0.1 mg, about 0.5 mg, about 1 mg, about 3 mg, about 5 mg, or about 10 mg. In aspects, a unit dose of a opioid agonist (e.g., naloxone) is about 1 mg to about 3 mg.

In aspects, a unit dose of an opioid antagonist (e.g., naloxone) is about 0.01 mg/kg body weight to about 1 mg/kg body weight. In aspects, a unit dose of an opioid antagonist (e.g., naloxone) is at least about 0.01 mg/kg body weight. In aspects, a unit dose of an opioid antagonist (e.g., naloxone) is at most about 1 mg/kg body weight. In aspects, a unit dose of an opioid antagonist (e.g., naloxone) is about 0.01 mg/kg body weight to about 0.05 mg/kg body weight, about 0.01 mg/kg body weight to about 0.1 mg/kg body weight, about 0.01 mg/kg body weight to about 0.5 mg/kg body weight, about 0.01 mg/kg body weight to about 1 mg/kg body weight, about 0.05 mg/kg body weight to about 0.1 mg/kg body weight, about 0.05 mg/kg body weight to about 0.5 mg/kg body weight, about 0.05 mg/kg body weight to about 1 mg/kg body weight, about 0.1 mg/kg body weight to about 0.5 mg/kg body weight, about 0.1 mg/kg body weight to about 1 mg/kg body weight, or about 0.5 mg/kg body weight to about 1 mg/kg body weight. In aspects, a unit dose of an opioid antagonist (e.g., naloxone) is about 0.01 mg/kg body weight, about 0.05 mg/kg body weight, about 0.1 mg/kg body weight, about 0.5 mg/kg body weight, or about 1 mg/kg body weight.

In aspects, a first pharmaceutical composition disclosed herein is in an immediate release dosage form. In aspects, the immediate release dosage form can release at least about 50%, 60%, 70%, 80%, or 90% of the therapeutic agent (e.g., doxapram and/or naloxone) within about 5 minutes after the administration. In aspects, the immediate release dosage form can release at least about 50%, 60%, 70%, 80%, or 90% of the therapeutic agent (e.g., doxapram and/or naloxone) within about 1 min to about 5 min. In aspects, the immediate release dosage form can release at least about 50%, 60%, 70%, 80%, or 90% of the therapeutic agent (e.g., doxapram and/or naloxone) within at least about 1 min. In some cases, the immediate release dosage form can release at least about 50%, 60%, 70%, 80%, or 90% of the therapeutic agent (e.g., doxapram and/or naloxone) within at most about 5 min. In aspects, the immediate release dosage form can release at least about 50%, 60%, 70%, 80%, or 90% of the therapeutic agent (e.g., doxapram and/or naloxone) within about 1 min to about 2 min, about 1 min to about 3 min, about 1 min to about 4 min, about 1 min to about 5 min, about 2 min to about 3 min, about 2 min to about 4 min, about 2 min to about 5 min, about 3 min to about 4 min, about 3 min to about 5 min, or about 4 min to about 5 min. In aspects, the immediate release dosage form can release at least about 50%, 60%, 70%, 80%, or 90% of the therapeutic agent (e.g., doxapram and/or naloxone) within about 1 min, about 2 min, about 3 min, about 4 min, or about 5 min.

Disclosed herein is also a second pharmaceutical composition comprising a sustained release dosage form of the respiratory stimulant. In aspects, the respiratory stimulant in the second pharmaceutical composition is the same respiratory stimulant as in the first pharmaceutical composition. In aspects, the respiratory stimulant in the second pharmaceutical composition is a different respiratory stimulant from the first pharmaceutical composition.

In aspects, a unit dose of a respiratory stimulant (e.g., doxapram) is about 10 mg to about 600 mg. In aspects, a unit dose of a respiratory stimulant (e.g., doxapram) is at least about 10 mg. In aspects, a unit dose of a respiratory stimulant (e.g., doxapram) is at most about 600 mg. In aspects, a unit dose of a respiratory stimulant (e.g., doxapram) is about 10 mg to about 50 mg, about 10 mg to about 100 mg, about 10 mg to about 200 mg, about 10 mg to about 300 mg, about 10 mg to about 500 mg, about 10 mg to about 600 mg, about 50 mg to about 100 mg, about 50 mg to about 200 mg, about 50 mg to about 300 mg, about 50 mg to about 500 mg, about 50 mg to about 600 mg, about 100 mg to about 200 mg, about 100 mg to about 300 mg, about 100 mg to about 500 mg, about 100 mg to about 600 mg, about 200 mg to about 300 mg, about 200 mg to about 500 mg, about 200 mg to about 600 mg, about 300 mg to about 500 mg, about 300 mg to about 600 mg, or about 500 mg to about 600 mg. In aspects, a unit dose of a respiratory stimulant (e.g., doxapram) is about 10 mg, about 50 mg, about 100 mg, about 200 mg, about 300 mg, about 500 mg, or about 600 mg. In aspects, a unit dose of a respiratory stimulant (e.g., doxapram) is about 100 mg to about 300 mg.

In aspects, a unit dose of a respiratory stimulant (e.g., doxapram) is about 0.2 mg/kg body weight to about 20 mg/kg body weight. In aspects, a unit dose of a respiratory stimulant (e.g., doxapram) is at least about 0.2 mg/kg body weight. In aspects, a unit dose of a respiratory stimulant (e.g., doxapram) is at most about 20 mg/kg body weight. In aspects, a unit dose of a respiratory stimulant (e.g., doxapram) is about 0.2 mg/kg body weight to about 0.5 mg/kg body weight, about 0.2 mg/kg body weight to about 1 mg/kg body weight, about 0.2 mg/kg body weight to about 2 mg/kg body weight, about 0.2 mg/kg body weight to about 5 mg/kg body weight, about 0.2 mg/kg body weight to about 10 mg/kg body weight, about 0.2 mg/kg body weight to about 20 mg/kg body weight, about 0.5 mg/kg body weight to about 1 mg/kg body weight, about 0.5 mg/kg body weight to about 2 mg/kg body weight, about 0.5 mg/kg body weight to about 5 mg/kg body weight, about 0.5 mg/kg body weight to about 10 mg/kg body weight, about 0.5 mg/kg body weight to about 20 mg/kg body weight, about 1 mg/kg body weight to about 2 mg/kg body weight, about 1 mg/kg body weight to about 5 mg/kg body weight, about 1 mg/kg body weight to about 10 mg/kg body weight, about 1 mg/kg body weight to about 20 mg/kg body weight, about 2 mg/kg body weight to about 5 mg/kg body weight, about 2 mg/kg body weight to about 10 mg/kg body weight, about 2 mg/kg body weight to about 20 mg/kg body weight, about 5 mg/kg body weight to about 10 mg/kg body weight, about 5 mg/kg body weight to about 20 mg/kg body weight, or about 10 mg/kg body weight to about 20 mg/kg body weight. In aspects, a unit dose of a respiratory stimulant (e.g., doxapram) is about 0.2 mg/kg body weight, about 0.5 mg/kg body weight, about 1 mg/kg body weight, about 2 mg/kg body weight, about 5 mg/kg body weight, about 10 mg/kg body weight, or about 20 mg/kg body weight.

In aspects, the concentration of a respiratory stimulant (e.g., doxapram) is about 1 mg/mL to about 20 mg/mL. In aspects, the concentration of a respiratory stimulant (e.g., doxapram) is about 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, or at least about 20 mg/mL. In aspects, the concentration of a respiratory stimulant (e.g., doxapram) is about 3 mg/mL. In aspects, the concentration of doxapram is about 3 mg/mL.

In aspects, the second pharmaceutical composition is in a sustained release dosage form. In aspects, the sustained release dosage form can release at least about 50%, 60%, 70%, 80%, or 90% of the therapeutic agent (e.g., doxapram) in a time frame from about 5 minutes to about 30 minutes after the administration. In aspects, the sustained release dosage form can release at least about 50%, 60%, 70%, 80%, or 90% of the therapeutic agent (e.g., doxapram) in a time frame about 5 min to about 10 min, about 5 min to about 15 min, about 5 min to about 20 min, about 5 min to about 25 min, about 5 min to about 30 min, about 10 min to about 15 min, about 10 min to about 20 min, about 10 min to about 25 min, about 10 min to about 30 min, about 15 min to about 20 min, about 15 min to about 25 min, about 15 min to about 30 min, about 20 min to about 25 min, about 20 min to about 30 min, or about 25 min to about 30 min after the administration. In aspects, the sustained release dosage form can release at least about 50%, 60%, 70%, 80%, or 90% of the therapeutic agent (e.g., doxapram) in a time frame about 10 min, about 15 min, about 20 min, about 25 min, or about 30 min.

In aspects, the second pharmaceutical composition may comprise a suspension agent. In aspects, the respiratory stimulant is encapsulated in the suspension agent. In aspects, the second pharmaceutical composition comprises about 0.1% of the suspension agent to about 90% of the suspension agent. In aspects, the second pharmaceutical composition comprises at least about 0.1% of the suspension agent. In aspects, the second pharmaceutical composition comprises at most about 90% of the suspension agent. In aspects, the second pharmaceutical composition comprises about 0.1% of the suspension agent to about 0.5% of the suspension agent, about 0.1% of the suspension agent to about 1% of the suspension agent, about 0.1% of the suspension agent to about 10% of the suspension agent, about 0.1% of the suspension agent to about 20% of the suspension agent, about 0.1% of the suspension agent to about 30% of the suspension agent, about 0.1% of the suspension agent to about 60% of the suspension agent, about 0.1% of the suspension agent to about 90% of the suspension agent, about 0.5% of the suspension agent to about 1% of the suspension agent, about 0.5% of the suspension agent to about 10% of the suspension agent, about 0.5% of the suspension agent to about 20% of the suspension agent, about 0.5% of the suspension agent to about 30% of the suspension agent, about 0.5% of the suspension agent to about 60% of the suspension agent, about 0.5% of the suspension agent to about 90% of the suspension agent, about 1% of the suspension agent to about 10% of the suspension agent, about 1% of the suspension agent to about 20% of the suspension agent, about 1% of the suspension agent to about 30% of the suspension agent, about 1% of the suspension agent to about 60% of the suspension agent, about 1% of the suspension agent to about 90% of the suspension agent, about 10% of the suspension agent to about 20% of the suspension agent, about 10% of the suspension agent to about 30% of the suspension agent, about 10% of the suspension agent to about 60% of the suspension agent, about 10% of the suspension agent to about 90% of the suspension agent, about 20% of the suspension agent to about 30% of the suspension agent, about 20% of the suspension agent to about 60% of the suspension agent, about 20% of the suspension agent to about 90% of the suspension agent, about 30% of the suspension agent to about 60% of the suspension agent, about 30% of the suspension agent to about 90% of the suspension agent, or about 60% of the suspension agent to about 90% of the suspension agent. In aspects, the second pharmaceutical composition comprises about 0.1% of the suspension agent, about 0.5% of the suspension agent, about 1% of the suspension agent, about 10% of the suspension agent, about 20% of the suspension agent, about 30% of the suspension agent, about 60% of the suspension agent, or about 90% of the suspension agent. In aspects, the second pharmaceutical composition comprises about 0.1% to about 30% of the suspension agent. In aspects, the second pharmaceutical composition comprises about 0.5% of the suspension agent. In aspects, the percent composition of the suspension agent is by weight (wt) of the second pharmaceutical composition. In aspects, the percent composition of the suspension agent is by volume (vol) of the second pharmaceutical composition.

In aspects, the suspension agent comprises a phospholipid solution. In aspects, the suspension agent comprises liposome. In aspects, the suspension agent comprises micelle. In aspects, the suspension agent comprises a lipid emulsion (e.g., INTRALIPID®), for example, comprising soybean oil, egg phospholipids, glycerin, or any combination thereof. In aspects, a lipid emulsion comprises soybean oil, egg phospholipids, glycerin, and water. In aspects, a lipid emulsion comprises 20% soybean oil, 1.2% egg phospholipids, 2.25% glycerin, and water. In aspects, the second pharmaceutical composition comprises a lipid emulsion. In aspects, the lipid emulsion encapsulates a respiratory stimulant (e.g., doxapram). In aspects, the lipid emulsion encapsulates a respiratory stimulant (e.g., doxapram), thereby forming a sustained release dosage form of the respiratory stimulant. In aspects, the lipid emulsion encapsulates doxapram, thereby forming the sustained release dosage form of doxapram.

In aspects, drug delivery performance provided by the dosage forms described herein can be evaluated using a standard USP type 2 or USP type 7 dissolution apparatus set to 37° C.±2° C. under the conditions described, for example, in the experimental examples provided herein. In aspects, the dissolution media may be selected from dissolution media known by those of skill in the art such as at least one of purified water, 0.1N HCl, simulated intestinal fluid, and others.

In aspects, the unit dose provided herein can be selected from liquid solutions, suspensions, tablets, mini-tablets, capsules, caplets, beads, pellets, granules, sachets, crystals, powders, sprays and combinations thereof.

In aspects, the amount of the unit dose can be about 1 ml to about 10 ml. In aspects, the amount of the unit dose can be at least about 1 ml. In aspects, the amount of the unit dose can be at most about 10 ml. In aspects, the amount of the unit dose can be about 1 ml to about 2 ml, about 1 ml to about 3 ml, about 1 ml to about 4 ml, about 1 ml to about 5 ml, about 1 ml to about 6 ml, about 1 ml to about 8 ml, about 1 ml to about 10 ml, about 2 ml to about 3 ml, about 2 ml to about 4 ml, about 2 ml to about 5 ml, about 2 ml to about 6 ml, about 2 ml to about 8 ml, about 2 ml to about 10 ml, about 3 ml to about 4 ml, about 3 ml to about 5 ml, about 3 ml to about 6 ml, about 3 ml to about 8 ml, about 3 ml to about 10 ml, about 4 ml to about 5 ml, about 4 ml to about 6 ml, about 4 ml to about 8 ml, about 4 ml to about 10 ml, about 5 ml to about 6 ml, about 5 ml to about 8 ml, about 5 ml to about 10 ml, about 6 ml to about 8 ml, about 6 ml to about 10 ml, or about 8 ml to about 10 ml. In aspects, the amount of the unit dose can be about 1 ml, about 2 ml, about 3 ml, about 4 ml, about 5 ml, about 6 ml, about 8 ml, or about 10 ml.

In aspects, the pharmaceutical compositions disclosed herein can comprise one or more pharmaceutically acceptable excipients including, but not limited to, binders, lubricants, glidants, disintegrants, diluents, coloring agents, suspension agents, and flavoring agents. Exemplary excipients are described in Remington, The Science and Practice of Pharmacy, 22nd Ed. 2013, which is incorporated herein by reference in its entirety. Other commonly used pharmaceutically acceptable excipients include, but are not limited to, water, magnesium stearate, starch, lactose, microcrystalline cellulose, stearic acid, sucrose, talc, silicon dioxide, gelatin, acacia, and dibasic calcium phosphate (Baldrick, P. (2000) Regul. Toxicol. Pharmacol. October 32(2):210; incorporated herein by reference.) Excipients can be combined with active ingredients for example to enhance appearance, improve stability, aid processing or aid disintegration after administration. In aspects, the excipients include, but are not limited to: natural, modified-natural or synthetic mono-, oligo- or polysaccharides, where oligo- and polysaccharides may or may not be physically or chemically crosslinked; natural, modified-natural or synthetic mono-, oligo- and polypeptides or proteins, where oligo- and polypeptides and proteins may or may not be physically or chemically crosslinked; synthetic oligomers and polymers that may or may not be physically or chemically crosslinked; monomeric, hydrophobic, hydrophilic or amphoteric organic molecules; inorganic salts or metals; and combinations thereof. In aspects, therapeutic agents used herein such as, for example, doxapram and/or naloxone, may be combined with any excipient(s) known in the art that allow tailoring performance during manufacturing, administration and/or its in vitro and in vivo performance.

In aspects, a material or materials which help suspend a composition of the invention in a liquid, for example water, for administration may be used. In aspects, suspension agents (or viscosity modifying agents) are exemplified by, but are not limited to, acacia, agar, alginic acid, bentonite, calcium stearate, carbomers, carboxymethylcellulose calcium, carboxymethylcellulose sodium, carrageenan, cellulose (powdered), ceratonia, colloidal silicon dioxide, dextrin, gelatin, guar gum, hectorite, hydrophobic colloidal silica, hydroxyethyl cellulose, hydroxyethylmethyl cellulose, hydroxypropyl cellulose, hypromellose, kaolin, magnesium aluminium silicate, maltitol solution, medium-chain triglycerides, methylcellulose, microcrystalline cellulose, phospholipids, polycarbophil, polyethylene glycol, polyoxyethylene sorbitan fatty acid esters, potassium alginate, povidone, propylene glycol alginate, saponite, sesame oil, sodium alginate, sodium starch glycolate, sorbitan esters, sucrose, tragacanth, vitamin E, polyethylene glycol, succinate, or xanthan gum, or combinations thereof.

In aspects, pH adjusting agents may be used in the invention and can include acids, bases and many of the compounds/salts found in U.S. Pat. No. 8,263,650. In aspects, the pH adjusting agent is an acid, for example, acetic, acetylsalicylic, barbital, barbituric, benzoic, benzyl penicillin, boric, caffeine, carbonic, citric, dichloroacetic, ethylenediaminetetra-acetic acid (EDTA), formic, glycerophosphoric, glycine, lactic, malic, mandelic, monochloroacetic, oxalic, phenobarbital, phenol, picric, propionic, saccharin, salicylic, sodium dihydrogen phosphate, succinic, sulfadiazine, sulfamerazine, sulfapyridine, sulfathiazole, tartaric, trichloroacetic, and the like, or inorganic acids such as hydrochloric, nitric, phosphoric or sulfuric, and the like. In aspects, the pH adjusting agent may be a pharmaceutically acceptable acid as listed in the “Handbook of Pharmaceutical Salts: Properties, Selection and Use” (P. Stahl; John Wiley & Sons, Aug. 4, 2008; included herein by reference).

### Device

In aspects, a device disclosed herein comprises at least one container. In aspects, the at least one container comprises a pharmaceutical composition provided herein. In aspects, a device comprises a first container comprising a first pharmaceutical composition provided herein and a second container comprising a second pharmaceutical composition provided herein. In aspects, the first pharmaceutical composition can comprise a respiratory stimulant and an opioid antagonist. In aspects, the second pharmaceutical composition can comprise a sustained release dosage form of the respiratory stimulant.

In aspects, a device disclosed herein can further comprise an auto-injector. In aspects, the device comprises a bifurcated auto-injector. In aspects, the auto-injector administers a pharmaceutical composition. In aspects, the auto-injector administers the first pharmaceutical composition and second pharmaceutical composition. In aspects, the auto-injector administers the first pharmaceutical composition and second pharmaceutical composition sequentially or concurrently. In aspects, the auto-injector administers the first pharmaceutical composition and second pharmaceutical composition sequentially. In aspects, the auto-injector administers the first pharmaceutical composition and second pharmaceutical composition concurrently. In aspects, the device can further comprise a housing, wherein the first and second containers are located within the housing.

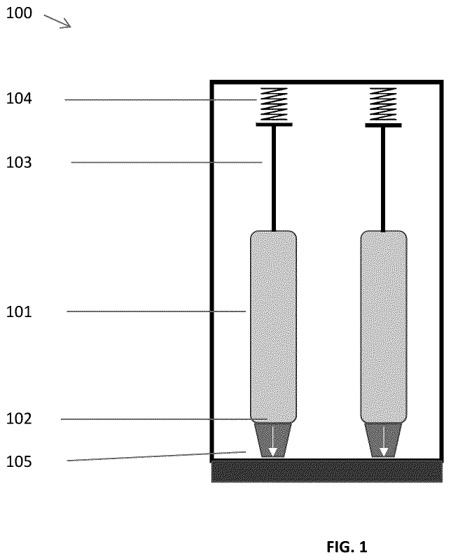

In aspects, the device comprising the auto-injector dispenses a predetermined dose of a medicament. In aspects, the device comprises a needle cover that is configured to move from a retracted position to an extended locked position. In aspects, the device comprises a first locking assembly to hold the needle cover in a locked retracted position and a second locking assembly to hold the needle cover in a locked extended position. An exemplary device (e.g., auto-injector) 100 is shown in FIG. 1. In aspects, the device 100 comprises a first and a second container (e.g., in form of syringe 101), each container equipped with a retracted and/or spring actuated needle 102, a plunger 103, and/or a compressed spring 104. In aspects, the device 100 comprises a trigger 105. In aspects, when the trigger 105 is activated, the compressed spring 104 is released, extending the plunger 103, actuating the needle 102, and pushing the medicament in syringe 101. In aspects, the medicament in syringe 101 is pushed into a subject in need thereof. In aspects, the medicament is pushed into a subject in need thereof via intramuscular injection. In aspects, the medicament is pushed into a subject in need thereof via subcutaneous injection. In aspects, the medicament is pushed into a subject in need thereof via intravenous injection. In aspects, the medicament is pushed into a subject in need thereof via intranasal administration.

In aspects, a device can be included in an emergency kit. In aspects, a device can be incorporated in an algorithm for Advanced Cardiac Life Support (ACLS). In aspects, a device can be used at home. In aspects, a device can be used at a hospital, an addiction treatment center, a mental health center, an urgent care, and the like.

### Methods

Disclosed here are methods for stimulating respiration. In aspects, a method for stimulating respiration comprises administering a pharmaceutical composition to a subject in need thereof. In aspects, a method for stimulating respiration comprises administering a first pharmaceutical composition and a second pharmaceutical composition to a subject in need thereof, wherein the first pharmaceutical composition comprises a respiratory stimulant and an opioid antagonist; and the second pharmaceutical composition comprises a sustained release dosage form of the respiratory stimulant. In aspects, the subject is a human. In aspects, the subject is a non-human animal. In aspects, the non-human animal is a non-human vertebrate, a non-human primate, a cetacean, a mammal, a reptile, a bird, an amphibian, or a fish. In aspects, the non-human animal is a bovine. In aspects, the non-human animal is a mustelid, a captive mustelid, a rodent, a captive rodent, a raptor, or a captive bird. In aspects, the non-human animal is a canine. In aspects, the non-human animal is a feline.

In aspects, a medicament, a pharmaceutical composition, and/or a formulation of the disclosure is administered. In aspects, a route for administration of a medicament, a pharmaceutical composition, and/or a formulation of the disclosure may be oral, sublingual, intravenous, nasal, inhalational, topical, buccal, rectal, pleural, peritoneal, vaginal, intramuscular, cutaneous, subcutaneous, transdermal, epidural, systemic, intratrachael, otic, intraocular, or intrathecal route. In aspects, the administration is via intramuscular injection. In aspects, the administration is via intramuscular injection. In aspects, the administration is via intranasal administration. In aspects, the administration is via subcutaneous injection. In in aspects, the administration is via a device provided herein. In some cases, the device can contain a fixed dose combination of the pharmaceutical compositions. In some cases, the pharmaceutical composition is pre-mixed.

In aspects, a subject in need thereof is administered one dose of a medicament, a pharmaceutical composition, and/or a formulation of the disclosure. In aspects, a subject in need thereof is administered two or more doses of medicament, a pharmaceutical composition, and/or a formulation of the disclosure. In aspects, a subject in need thereof is administered 1, 2, 3, 4, 5, 6, 7, 8, 9, or up to about 10 doses of a medicament, a pharmaceutical composition, and/or a formulation of the disclosure. In aspects, a subject in need thereof is administered about 1 to about 10 doses, about 1 to about 6 doses, about 2 to about 6 doses, about 2 to about 10 doses, about 1 to about 3 doses, or about 1 to about 4 doses of a medicament, a pharmaceutical composition, and/or a formulation of the disclosure. In aspects, one or more of a dose are administered using a device disclosed herein.

In aspects, a subject in need thereof is administered a medicament, a pharmaceutical composition, and/or a formulation of the disclosure, or a device comprising the same. In aspects, a subject in need thereof is in need of respiration stimulation and/or displays abnormal respiration. In aspects, a subject in need thereof is in need of respiration stimulation. In aspects, a subject in need thereof displays abnormal respiration. In aspects, a subject in need thereof has drug-induced postanesthesia respiratory depression or apnea, respiratory or central nervous system depression due to drug overdosage, e.g., caused by one or more of an opioid agonist, benzodiazepine, barbiturate or gabapentinoid, acute respiratory insufficiency superimposed on chronic obstructive pulmonary disease (COPD), cardiopulmonary arrest, or a combination thereof. In aspects, the subject has drug-induced postanesthesia respiratory depression or apnea. In aspects, the subject has respiratory or central nervous system depression due to drug overdosage, e.g., caused by one or more of an opioid agonist, benzodiazepine, barbiturate or gabapentinoid. In aspects, the subject has acute respiratory insufficiency superimposed on chronic obstructive pulmonary disease (COPD). In aspects, the subject is suffering from cardiopulmonary arrest. In aspects, the subject has one or more of the following conditions: respiratory depression, sedation, hypotension, adverse central nervous system effects, adverse cardiac effects, fever, chest pain, rapid breathing, labored breathing increased heart rate, increased blood pressure, sweating, convulsions, tremors, rise in body temperature, cardiovascular collapse, anxiety, agitation, and combative behavior, or any combination thereof.

In aspects, a drug overdosage is caused by one or more opioids, for example, morphine, codeine, thebaine, oripavine, diacetylmorphine, 2,4-dinitrophenylmorphine, methylenedioxydimethylamphetamine, ehlomaltrexamine, dihydromorphine, hydromorphinol, nicomorphine, dipropanoylmorphine, desomorphine, acetylproprionylmorphine, methyldesorphine, N-phenethylnormorphine, 14-hydroxydihydrocodeine (RAM-318), 7, 8-dihydro-14-hydroxy-N-phenethylnormorphme (RAM-378), dibenzoylmorphine, diacetyldihydromorphine, dibenzoylmorphine, 6-monoacetylcodeine (6-MAC), acetyldihydrocodeine, dihydroeodeme, nalbuphine, nicocodeine, nicodicodeine, oxymorphazone, 1-iodomorphine, morphine-6-glycuronide (M6G), 6-monoacetylmorphine (6-MAM), norcodeine, normorphine, genomorphine, dextrallorphan (DXA), cyclorphan, dihydroheterocodeine, pholcodine, myrophine, 14-cinnamoyloxycodeinone, 14-ethoxymetopon, 14-methoxymetopon, 14-phenylpropoxymetopon (PPOM), 7-spiroindanyloxymorphone, acetylmorphone, codeinone, conorphone, eodoxime, thebacon, metopon, N-phenethyl-14-ethoxymetopon, morphinone, benzylmorphine, codeine methylbromide, ethylmorphine, heterocodeine, hydromorphone, hydrocodone, oxycodone, oxymorphone, pentamorphone, semorphone, chloromorphide, ethylmorphine, buprenorphine, fentanyl, alphamethylfentanyl, alfentanil, sufentanil, remifentanil, carfentanyl, ohmefentanyl, pethidine, ketobemidone, desmethylprodine (MPPP), allylprodine, prodine, 1-methyl-4-phenyl-4-propionoxypiperidine (PEPAP), propoxyphene, dextropropoxyphene, dextromoramide, bezitramide, piritramide, levorphanol, methadone, dipipanone, levomethadyl acetate (LAAM), difenoxin, diphenoxylate, loperamide, dezoeine, pentazocine, phenazocine, dihydroetorphine, etorphine, butorphanol, nalbuphine, levomethorphan, levophenacylmorphan, norlevorphanol, phenomorphan, furethylnorlevorphanol, xorphanol, butorphanol, cyprodime, drotebanol, 7-PET, acetorphine, BU-48, cyprenorphine, norbuprenorphine, lefetamine, meptazinol, mitragynine, tilidine, tramadol, tapentadol, dextropropoxyphene, endorphins, enkephalins, dynorphins, and endomorphins, and combinations thereof. In aspects, the opioid is oxymorphone. In aspects, the opioid is oxycodone.

In aspects, a drug overdosage is caused by one or more benzodiazepine drugs including, but not limited to, alprazolam, brotizolam, chlordiazepoxide, clobazam, clonazepam, clorazepam, demoxazepam, diazepam, flumazenil, flurazepam, halazepam, midazolam, nordazepam, medazepam, nitrazepam, oxazepam, lorazepam, prazepam, quazepam, triazolam, temazepam, and loprazolam, and combinations thereof.

The drug overdosage can be caused by one or more barbiturate drugs selected from the group consisting of amobarbital, amobarbital sodium, aprobarbital, butabarbital sodium, hexobarbital sodium, mephobarbital, metharbital, methohexital sodium, pentobarbital sodium, phenobarbital, phenobarbital sodium, secobarbital sodium, taibutal, thiamylal sodium, and thiopental sodium, and combinations thereof.

In aspects, a drug overdosage is caused by one or more insomnia drugs such as a non-benzodiazepine hypnotic, a direct GABA agonist, a positive allosteric modulator of GABA receptors, a histamine receptor antagonist, or a histamine receptor inverse agonist, or combinations thereof. In aspects, the insomnia drug can be, for example, zolpidem, zopiclone, eszopiclone, zaleplon, gaboxadol, indiplon, or abecamil, or combinations thereof.

### Assessments

In aspects, a subject is assessed. An assessment can occur at any point before, during, or after administration with a pharmaceutical composition (e.g., comprising doxapram). In aspects, an assessment is performed before administration. In aspects, an assessment is performed during administration. In aspects, an assessment is performed post administration. In aspects, a subject is assessed by the minute, hourly, daily, weekly, monthly, or yearly.

In aspects, an assessment comprises determining a concentration of a pharmaceutical composition (e.g., comprising doxapram) in a subject in need thereof post administration, or a sample taken therefrom. In aspects, an assessment comprises a pharmacokinetic (PK) assessment. In aspects, a sample is a blood sample or a plasma sample, or a combination of both. In aspects, a suitable assay to measure pharmacokinetics may comprise electrochemiluminescence (ECL) assay, a bead-based assay, a cell-based assay, and combinations thereof. In aspects, a sample may comprise plasma and the plasma is assessed for a pharmaceutical composition (e.g., comprising doxapram) concentration by measuring: maximum plasma concentration (Cmax), area under the concentration-time curve (AUC), clearance (CL), or terminal elimination half-life (t^{1/2}), or combinations thereof.

In aspects, a PK assessment to a pharmaceutical composition (e.g., comprising doxapram) can occur about 0 minutes, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, or at least about 60 minutes post administration. In aspects, a PK assessment to a pharmaceutical composition (e.g., comprising doxapram) can be assessed about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or at least about 24 hours post administration. In aspects, a PK assessment to a pharmaceutical composition (e.g., comprising doxapram) can occur about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a PK assessment to a pharmaceutical composition (e.g., comprising doxapram) can occur on about 0 minutes, 5 minutes, 10 minutes, 15 minutes, 20 minutes, 25 minutes, 30 minutes, 35 minutes, 40 minutes, 45 minutes, 50 minutes, 55 minutes and 60 minutes (or 1 hour) post administration. In aspects, a PK assessment to a pharmaceutical composition (e.g., comprising doxapram) can occur about 7 days (or 1 week) post administration. In aspects, a PK assessment can occur at any point before, during, or after administration with a pharmaceutical composition (e.g., comprising doxapram).

In aspects, a subject administered a pharmaceutical composition of the disclosure (e.g., comprising doxapram) is assessed for Cmax of the pharmaceutical composition. In aspects, a subject administered a pharmaceutical composition of the disclosure (e.g., comprising doxapram) reaches Cmax in less than about 1 minute, 2 minutes, 3 minutes, 4 minutes or 5 minutes. In aspects, a subject administered a pharmaceutical composition of the disclosure (e.g., comprising doxapram) reaches Cmax in about 1 minute to about 180 minutes. In aspects, a subject administered a pharmaceutical composition of the disclosure (e.g., comprising doxapram) reaches Cmax in about 1 minutes, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 60 minutes, 61 minutes, 62 minutes, 63 minutes, 64 minutes, 65 minutes, 66 minutes, 67 minutes, 68 minutes, 69 minutes, 70 minutes, 71 minutes, 72 minutes, 73 minutes, 74 minutes, 75 minutes, 76 minutes, 77 minutes, 78 minutes, 79 minutes, 80 minutes, 81 minutes, 82 minutes, 83 minutes, 84 minutes, 85 minutes, 86 minutes, 87 minutes, 88 minutes, 89 minutes, 90 minutes, 91 minutes, 92 minutes, 93 minutes, 94 minutes, 95 minutes, 96 minutes, 97 minutes, 98 minutes, 99 minutes, 100 minutes, 101 minutes, 102 minutes, 103 minutes, 104 minutes, 105 minutes, 106 minutes, 107 minutes, 108 minutes, 109 minutes, 110 minutes, 111 minutes, 112 minutes, 113 minutes, 114 minutes, 115 minutes, 116 minutes, 117 minutes, 118 minutes, 119 minutes, 120 minutes, 121 minutes, 122 minutes, 123 minutes, 124 minutes, 125 minutes, 126 minutes, 127 minutes, 128 minutes, 129 minutes, 130 minutes, 131 minutes, 132 minutes, 133 minutes, 134 minutes, 135 minutes, 136 minutes, 137 minutes, 138 minutes, 139 minutes, 140 minutes, 141 minutes, 142 minutes, 143 minutes, 144 minutes, 145 minutes, 146 minutes, 147 minutes, 148 minutes, 149 minutes, 150 minutes, 151 minutes, 152 minutes, 153 minutes, 154 minutes, 155 minutes, 156 minutes, 157 minutes, 158 minutes, 159 minutes, 160 minutes, 161 minutes, 162 minutes, 163 minutes, 164 minutes, 165 minutes, 166 minutes, 167 minutes, 168 minutes, 169 minutes, 170 minutes, 171 minutes, 172 minutes, 173 minutes, 174 minutes, 175 minutes, 176 minutes, 177 minutes, 178 minutes, 179 minutes, or up to about 180 minutes. In aspects, a subject administered a pharmaceutical composition of the disclosure (e.g., comprising doxapram) reaches Cmax in about 1 minute to about 5 minutes, about 1 minute to about 15 minutes, about 5 minutes to about 15 minutes, about 5 minutes to about 30 minutes, about 15 minutes to about 30 minutes, about 1 minute to about 45 minutes, about 5 minutes to about 45 minutes, about 5 minutes to about 120 minutes, about 5 minutes to about 180 minutes, about 45 minutes to about 180 minutes, about 45 minutes to about 120 minutes, about 5 minutes to about 60 minutes, about 30 minutes to about 60 minutes, about 30 minutes to about 120 minutes, about 60 minutes to about 150 minutes, or about 60 minutes to about 180 minutes. In aspects, a subject administered a pharmaceutical composition of the disclosure (e.g., comprising doxapram) reaches Cmax in about 1 minute to about 5 minutes. In aspects, a subject administered a pharmaceutical composition of the disclosure (e.g., comprising doxapram) reaches Cmax in about 15 minutes to about 30 minutes. In aspects, a subject administered a pharmaceutical composition of the disclosure (e.g., comprising doxapram) reaches Cmax in about 5 minutes to about 45 minutes.

In aspects, a subject administered a pharmaceutical composition of the disclosure (e.g., comprising doxapram) reaches Cmax and maintains a plasma concentration of more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or up to about 100% Cmax of the pharmaceutical composition for at least about 1 hour. In aspects, a subject administered a pharmaceutical composition of the disclosure (e.g., comprising doxapram) reaches Cmax and maintains a plasma concentration of more than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or up to about 100% of said Cmax of the pharmaceutical composition for up to about 1 hour. In aspects, a subject administered a pharmaceutical composition of the disclosure (e.g., comprising doxapram) reaches Cmax and maintains a plasma concentration of more than about 5% of said Cmax for at least about 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, or at least about 60 minutes. In aspects, a subject administered a pharmaceutical composition of the disclosure (e.g., comprising doxapram) reaches Cmax and maintains a plasma concentration of more than about 5% of said Cmax for at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or up to about 24 hours. In aspects, a subject administered a pharmaceutical composition of the disclosure (e.g., comprising doxapram) reaches Cmax and maintains a plasma concentration of more than about 5% of said Cmax after a single dose of the pharmaceutical composition. In aspects, a subject administered a pharmaceutical composition of the disclosure (e.g., comprising doxapram) reaches Cmax and maintains a plasma concentration of more than about 5% of said Cmax after two or more doses of the pharmaceutical composition. In aspects, a subject administered a pharmaceutical composition of the disclosure (e.g., comprising doxapram) reaches Cmax and maintains a plasma concentration of more than about 5% of said Cmax after a single dose of the pharmaceutical composition by intramuscular injection. In aspects, a subject administered a pharmaceutical composition of the disclosure (e.g., comprising doxapram) reaches Cmax and maintains a plasma concentration of more than about 5% of said Cmax after two or more doses of the pharmaceutical composition by intramuscular injection.

### NUMBERED EMBODIMENTS

Notwithstanding the appended claims, the following numbered embodiments also form part of the instant disclosure.

### Embodiment Set 1

1. device, comprising: a first container comprising a first pharmaceutical composition, wherein the first pharmaceutical composition comprises a respiratory stimulant and an opioid antagonist; and a second container comprising a second pharmaceutical composition, wherein the second pharmaceutical composition comprises a sustained release dosage form of the respiratory stimulant.

2. The device of embodiment 1, wherein the respiratory stimulant comprises doxapram, nikethamide, pentetrazol, etamivan, bemegride, prethcamide, almitrine, dimefline, or mepixanox.

3. The device of embodiment 1 or 2, wherein the opioid antagonist comprises naloxone, naltrexone, nalbuphine, butorphanol, pentazocine, diprenorphine, or dihydroetorphine.

4. The device of embodiment 1, wherein the first pharmaceutical composition comprises doxapram and naloxone, and the second pharmaceutical composition comprises the sustained release dosage form of doxapram.

5. The device of any one of embodiments 1-4, wherein the first pharmaceutical composition comprises a unit dose of doxapram ranging from about 5 to about 300 mg.

6. The device of embodiment 5, wherein the unit dose of doxapram is about 50 or 150 mg.

7. The device of any one of embodiments 1-6, wherein the first pharmaceutical composition comprises a unit dose of naloxone ranging from about 0.05 to about 10 mg.

8. The device of embodiment 7, wherein the unit dose of naloxone is about 1 or 3 mg.

9. The device of any one of embodiments 1-8, wherein the second pharmaceutical composition comprises a unit dose of doxapram ranging from about 10 to about 600 mg.

10. The device of embodiment 9, wherein the unit dose of doxapram is about 100 or 300 mg.

11. The device of any one of embodiments 1-10, wherein the first pharmaceutical composition comprises a unit dose of doxapram ranging from about 0.1 to about 10 mg/kg body weight.

12. The device of any one of embodiments 1-11, wherein the first pharmaceutical composition comprises a unit dose of naloxone ranging from about 0.01 to about 1 mg/kg body weight.

13. The device of any one of embodiments 1-12, wherein the second pharmaceutical composition comprises a unit dose of doxapram ranging from about 0.2 to about 20 mg/kg body weight.

14. The device of any one of embodiments 1-13, wherein the second pharmaceutical composition further comprises a suspension agent.

15. The device of embodiment 14, wherein the respiratory stimulant is encapsulated in the suspension agent.

16. The device of any one of embodiments 14-15, wherein the suspension agent ranges from about 0.1% to about 90% (wt) of the second pharmaceutical composition.

17. The device of embodiment 16, wherein the suspension agent is about 0.5% (wt) of the second pharmaceutical composition.

18. The device of any one of embodiments 14-17, wherein the suspension agent comprises a phospholipid solution.

19. The device of embodiment 18, wherein the suspension agent comprises a lipid emulsion.

20. The device of embodiment 19, wherein the lipid emulsion comprises liposome or micelle.

21. The device of embodiment 19, wherein the lipid emulsion comprises soybean oil, egg phospholipids, glycerin, or any combination thereof.

22. The device of embodiment any one of embodiments 1-21, further comprising an auto-injector suitable for administering the first and second pharmaceutical compositions.

23. The device of embodiment 22, wherein the auto-injector is suitable for administering the first and second pharmaceutical compositions sequentially or concurrently.

24. The device of embodiment any one of embodiments 1-23, further comprising a housing, wherein the first and second containers are located within the housing.

25. A method for stimulating respiration, comprising administering a first pharmaceutical composition and a second pharmaceutical composition to a subject in need thereof, wherein the first pharmaceutical composition comprises a respiratory stimulant and an opioid antagonist; and the second pharmaceutical composition comprises a sustained release dosage form of the respiratory stimulant.

26. The method of embodiment 25, wherein the subject has drug-induced postanesthesia respiratory depression or apnea.

27. The method of embodiment 25, wherein the subject has respiratory or central nervous system depression due to drug overdosage.

28. The method of embodiment 25, wherein the subject has acute respiratory insufficiency superimposed on chronic obstructive pulmonary disease.

29. The method of embodiment 25, wherein the subject is suffering from cardiopulmonary arrest.

30. The method of any one of embodiments 25-29, wherein the first pharmaceutical composition is encompassed in a first container of a device, wherein the second pharmaceutical composition is encompassed in a second container of the device, and wherein the device is suitable for administering the first and second pharmaceutical compositions.

31. The method of embodiment 30, wherein the first and second pharmaceutical compositions are administered using an auto-injector.

32. The method of any one of embodiments 30-31, wherein the first and second pharmaceutical compositions are administered sequentially or concurrently.

33. The method of any one of embodiments 30-32, wherein the device further comprises a housing, wherein the first and second containers are located within the housing.

34. The method of any one of embodiments 25-33, wherein the respiratory stimulant comprises doxapram, nikethamide, pentetrazol, etamivan, bemegride, prethcamide, almitrine, dimefline, or mepixanox.

35. The method of any one of embodiments 25-34, wherein the opioid antagonist comprises naloxone, naltrexone, nalbuphine, butorphanol, pentazocine, diprenorphine, or dihydroetorphine.

36. The method of any one of embodiments 30-35, wherein the first pharmaceutical composition comprises doxapram and naloxone, and the second pharmaceutical composition comprises the sustained release dosage form of doxapram.

37. The method of any one of embodiments 30-36, wherein the first pharmaceutical composition comprises a unit dose of doxapram ranging from about 5 to about 300 mg.

38. The method of embodiment 37, wherein the unit dose of doxapram is about 50 or 150 mg.

39. The method of any one of embodiments 30-38, wherein the first pharmaceutical composition comprises a unit dose of naloxone ranging from about 0.05 to about 10 mg.

40. The method of embodiment 39, wherein the unit dose of naloxone is about 1 or 3 mg.

41. The method of any one of embodiments 30-40, wherein the second pharmaceutical composition comprises a unit dose of doxapram ranging from about 10 to about 600 mg.

42. The method of embodiment 41, wherein the unit dose of doxapram is about 100 or 300 mg.

43. The method of any one of embodiments 30-42, wherein the first pharmaceutical composition comprises a unit dose of doxapram ranging from about 0.1 to about 10 mg/kg body weight.

44. The method of any one of embodiments 30-43, wherein the first pharmaceutical composition comprises a unit dose of naloxone ranging from about 0.01 to about 1 mg/kg body weight.

45. The method of any one of embodiments 30-44, wherein the second pharmaceutical composition comprises a unit dose of doxapram ranging from about 0.2 to about 20 mg/kg body weight.

46. The method of any one of embodiments 30-45, wherein the second pharmaceutical composition further comprises a suspension agent.

47. The method of embodiment 46, wherein the respiratory stimulant is encapsulated in the suspension agent.

48. The method of any one of embodiments 46-47, wherein the suspension agent ranges from about 0.1% to about 30% of the second pharmaceutical composition.

49. The method of embodiment 48, wherein the suspension agent is about 0.5% of the second pharmaceutical composition.

50. The method of any one of embodiments 46-49, wherein the suspension agent comprises a phospholipid solution.

51. The method of embodiment 50, wherein the suspension agent comprises a lipid emulsion.

52. The method of embodiment 51, wherein the lipid emulsion comprises liposome or micelle.

53. The method of embodiment 51, wherein the lipid emulsion comprises soy bean oil, egg phospholipids, glycerin, or any combination thereof.

54. A pharmaceutical composition comprising doxapram and naloxone, wherein the pharmaceutical composition comprises a unit dose of doxapram ranging from about 5 to about 300 mg and a unit dose of naloxone ranging from about 0.05 to about 10 mg.

55. The pharmaceutical composition of embodiment 54, wherein the unit dose of doxapram is about 50 or 150 mg.

56. The pharmaceutical composition of any one of embodiments 54-55, wherein the unit dose of naloxone is about 1 or 3 mg.

57. The pharmaceutical composition of any one of embodiments 54-56, wherein the unit dose of doxapram ranging from about 0.1 to about 10 mg/kg body weight.

58. The pharmaceutical composition of any one of embodiments 54-57, wherein the unit dose of naloxone ranging from about 0.01 to about 1 mg/kg body weight.

59. A pharmaceutical composition, wherein the pharmaceutical composition comprises a sustained release dosage form of doxapram comprising a lipid emulsion.

60. The pharmaceutical composition of embodiment 59, wherein the unit dose of doxapram is about 10 to about 600 mg.

61. The pharmaceutical composition of embodiment 60, wherein the unit dose of doxapram is about 100 or 300 mg.

62. The pharmaceutical composition of any one of embodiments 59-61, wherein the unit dose of doxapram ranging from about 0.2 to about 20 mg/kg body weight.

63. The pharmaceutical composition of any one of embodiments 59-62, wherein the lipid emulsion ranges from about 0.1% to about 30% of the second pharmaceutical composition.

64. The pharmaceutical composition of any one of embodiments 59-63, wherein the lipid emulsion comprises liposome or micelle.

65. The pharmaceutical composition of any one of embodiments 59-64, wherein the lipid emulsion comprises soy bean oil, egg phospholipids, glycerin, or any combination thereof.

66. A method for stimulating respiration, comprising administering the pharmaceutical composition of any one of embodiments 54-65 to a subject in need thereof.

67. The method of embodiment 66, wherein the subject has drug-induced postanesthesia respiratory depression or apnea.

68. The method of embodiment 66, wherein the subject has respiratory or central nervous system depression due to drug overdosage.

69. The method of embodiment 66, wherein the subject has acute respiratory insufficiency superimposed on chronic obstructive pulmonary disease.

70. The method of embodiment 66, wherein the subject is suffering from cardiopulmonary arrest.

### Embodiment Set 2

1. A pharmaceutical composition comprising doxapram, wherein the pharmaceutical composition comprises: i) a unit dose of doxapram from about 5 mg to about 600 mg; and ii) a lipid emulsion.

2. The pharmaceutical composition of embodiment 1, wherein the unit dose of doxapram is about 50 to about 150 mg.

3. The pharmaceutical composition of embodiment 2, wherein the lipid emulsion comprises soy bean oil, egg phospholipids, glycerin, or any combination thereof.

4. The pharmaceutical composition of embodiment 3, wherein the lipid emulsion ranges from about 0.1% to about 30% (wt) of the pharmaceutical composition.

5. The pharmaceutical composition of embodiment 4, wherein the doxapram is encapsulated in the lipid emulsion as a sustained release dosage form of doxapram.

6. The pharmaceutical composition of embodiment 5, wherein the doxapram is about 3 mg/mL.

7. The pharmaceutical composition of embodiment 6, wherein the lipid emulsion is at a concentration of about 1.5% (wt) of the pharmaceutical composition, wherein the lipid emulsion comprises 20% soybean oil, 1.2% egg phospholipids, 2.25% glycerin, and water, and wherein the doxapram is either in racemate or (S)-enantiomer form.

8. A device, comprising at least one container, the at least one container comprising a pharmaceutical composition comprising a sustained release dosage form of a respiratory stimulant and a lipid emulsion.

9. The device of embodiment 8, wherein the at least one container comprises the pharmaceutical composition of embodiment 7.

10. The device of embodiment 8, comprising: i) a first container comprising a first pharmaceutical composition, wherein the first pharmaceutical composition comprises the respiratory stimulant and an opioid antagonist; and ii) a second container comprising a second pharmaceutical composition, wherein the second pharmaceutical composition comprises the sustained release dosage form of the respiratory stimulant and the lipid emulsion.

11. The device of embodiment 10, wherein the first pharmaceutical composition comprises a unit dose of the opioid antagonist of about 0.05 mg to about 10 mg and a unit dose of the respiratory stimulant of about 5 mg to about 300 mg and the second pharmaceutical composition comprises a unit dose of the respiratory stimulant of about 10 mg to about 600 mg.

12. The device of embodiment 11, wherein the first pharmaceutical composition comprises a unit dose of the opioid antagonist of about 1 mg to about 3 mg and a unit dose of the respiratory stimulant of about 50 mg to about 150 mg and the second pharmaceutical composition comprises a unit dose of the respiratory stimulant of about 100 mg to about 300 mg.

13. The device of embodiment 12, wherein the first pharmaceutical composition comprises a unit dose of the opioid antagonist of about 0.01 mg/kg body weight to about 1 mg/kg body weight and a unit dose of the respiratory stimulant of about 0.1 mg/kg body weight to about 10 mg/kg body weight and the second pharmaceutical composition comprises a unit dose of the respiratory stimulant of about 0.2 mg/kg body weight to about 20 mg/kg body weight.

14. The device of embodiment 13, wherein the lipid emulsion encapsulates the respiratory stimulant, and wherein the lipid emulsion ranges from about 0.1% to about 90% (wt) of the second pharmaceutical composition.

15. The device of embodiment 14, wherein the lipid emulsion is about 0.1% to about 30% (wt) of the second pharmaceutical composition.

16. The device of embodiment 15, wherein the lipid emulsion comprises soybean oil, egg phospholipids, glycerin, or any combination thereof.

17. The device of embodiment 16, wherein the respiratory stimulant comprises doxapram, nikethamide, pentetrazol, etamivan, bemegride, prethcamide, almitrine, dimefline, or mepixanox and wherein the opioid antagonist comprises naloxone, naltrexone, nalbuphine, butorphanol, pentazocine, diprenorphine, or dihydroetorphine.

18. The device of embodiment 17, wherein the first pharmaceutical composition comprises doxapram and naloxone, and the second pharmaceutical composition comprises the sustained release dosage form of doxapram.

19. A device, comprising: i) a first container comprising a first pharmaceutical composition, wherein the first pharmaceutical composition comprises doxapram and naloxone; and ii) a second container comprising a second pharmaceutical composition, wherein the second pharmaceutical composition comprises a sustained release dosage form of doxapram and a lipid emulsion, wherein the first pharmaceutical composition comprises a unit dose of naloxone of about 1 mg to about 3 mg and a unit dose of doxapram of about 50 mg to about 150 mg and the second pharmaceutical composition comprises a unit dose of doxapram of about 100 mg to about 300 mg, and wherein the lipid emulsion is about 0.1% to about 30% (wt) of the second pharmaceutical composition.

20. The device of embodiment 19, wherein the device is suitable for administering the first and second pharmaceutical compositions.

21. The device of embodiment 20, wherein the first and second pharmaceutical compositions are administered using an auto-injector, wherein the administration occurs sequentially or concurrently.

22. A method for stimulating respiration, comprising administering a pharmaceutical composition to a subject in need thereof, wherein the pharmaceutical composition comprises: i) a unit dose of doxapram from about 5 mg to about 600 mg; and ii) a lipid emulsion.

23. The method of embodiment 22, wherein the lipid emulsion comprises soy bean oil, egg phospholipids, glycerin, or any combination thereof.

24. The method of embodiment 23, wherein the lipid emulsion ranges from about 0.1% to about 30% (wt) of the pharmaceutical composition.

25. The method of embodiment 24, wherein the lipid emulsion ranges from about 0.5% to about 1.5% (wt) of the pharmaceutical composition.

26. The method of embodiment 25, wherein the subject has drug-induced post-anesthesia respiratory depression, apnea, respiratory or central nervous system depression due to drug overdose, acute respiratory insufficiency superimposed on chronic obstructive pulmonary disease (COPD), or cardiopulmonary arrest.

27. The method of embodiment 22, comprising a first pharmaceutical composition and a second first pharmaceutical composition, wherein the first pharmaceutical composition comprises a unit dose of naloxone of about 1 mg to about 3 mg and a unit dose of doxapram of about 50 mg to about 150 mg and the second pharmaceutical composition comprises a unit dose of doxapram of about 100 mg to about 300 mg, and wherein the lipid emulsion is about 0.1% to about 30% (wt) of the second pharmaceutical composition.

28. The method of embodiment 27, wherein the subject has respiratory or central nervous system depression due to drug overdose.

29. The method of embodiment 28, wherein the first pharmaceutical composition comprises a unit dose of naloxone of about 0.01 mg/kg body weight to about 1 mg/kg body weight and a unit dose of doxapram of about 0.1 mg/kg body weight to about 10 mg/kg body weight and the second pharmaceutical composition comprises a unit dose of doxapram of about 0.2 mg/kg body weight to about 20 mg/kg body weight.

30. The method of embodiment 29, wherein the doxapram reaches a maximum plasma concentration (Cmax) in about 15 minutes to about 30 minutes and maintains a plasma concentration of more than about 5% of said Cmax for at least once hour, after a single dose of said pharmaceutical composition by intramuscular injection.

### EXAMPLES

### Example 1—Preparation of the First Pharmaceutical Composition (Doxapram and Naloxone)

When doxapram and naloxone were first combined in their current finished dosage form, a precipitate was formed. Thus, an improved process was developed to provide a doxapram/naloxone pharmaceutical composition without causing a precipitate. The detailed steps of the improved process include:
1) 7.5 ml of final dose form doxapram (20 mg/ml in aqueous solution with benzyl alcohol) was measured using a sterile medical syringe and placed on a room temperature glass plate.
2) The glass plate was gently heated to 95 F using an electric hotplate with even heat distribution to minimize the chance of higher temperatures on various parts of the surface. Temperature was monitored by infrared thermometer.
3) The dehydration process was closely monitored by gently tilting the plate side to side to evaluate the presence of liquid and the odor was evaluated for the presence of evaporating benzyl alcohol detected by its characteristic aromatic nature.
4) The dehydration process took less than 10 min, at which time the remaining substance on the surface was no longer subjected to movement when the plate was tilted and the aromatic benzyl alcohol vapors were no longer present.
5) The remaining residue was shiny in appearance and mildly sticky when touched.
6) The residue was then removed using a clean razorblade. The residue then took on the appearance of a very slightly off-white, slightly sticky crystalline substance.
7) The substance was then transferred to clean wax paper.
8) The wax paper was used to transfer the substrate to a test-tube containing 1 ml of room temperature distilled water and was mildly agitated to increase the rate of dissolution.
9) Additional aliquots of room temperature distilled water were added to the test-tube until the substrate was completely dissolved. This occurred between 2.5 ml and 3 ml. Alternatively, a lower volume of room temperature distilled water could be used by using mild heat and a mechanical stirring device.
10) On a separate, clean, room temperature plate, 3 ml of 1 mg/ml naloxone was placed using a
clean, sterile syringe in the same method as step 1, above.
11) The glass plate was heated, and the temperature was monitored in the same manner as step 2, above.
12) The liquid was closely monitored for dehydration using the same tilting method as step 3, above. Importantly, there was no aromatic vapor from this solution as it did not contain benzyl alcohol.
13) When dehydrated, the remaining substance was less shiny than the doxapram and not sticky. It
was removed using a clean razorblade as in step 6, above.
14) The substance was then transferred to wax paper as in step 7, above.
15) The wax paper was then used to transfer the substance to the previously produced solution from step 9, where the contents dissolved completely.
16) For the pediatric/canine dosage, the process was the same save for the following differences:
   a) 2.5 ml of FDF doxapram was placed on the room temperature plate, instead of 7.5 ml
   b) The resultant substance from (a) was added to 1 ml of distilled water.
   c) 1 ml of FDF naloxone 1 mg/ml was placed on the room temperature plate, instead of 3 ml
   d) The resultant substance from (c) was added to the solution created in (b).

For adult dosages, 150 mg of doxapram and 3 mg of Naloxone is used in 3 mL aqueous solution. For pediatric or canine dosages, 50 mg of doxapram and 1 mg of Naloxone is used in a 1 mL aqueous solution.

### Example 2—Preparation of the Second Pharmaceutical Composition (Doxapram Sustained Release Dosage Form)

To create the sustained release doxapram solution, doxapram is combined with a lipid emulsion at a concentration of 0.5 wt %. The lipid emulsion can be a sterile fat emulsion comprising 20% soybean oil, 1.2% egg yolk phospholipids, 2.25% glycerin, and water. Sodium hydroxide can be added to adjust the pH of the lipid emulsion to about 8.0.
1) 15 ml of final dose form (FDF) doxapram (20 mg/ml in aqueous solution with benzyl alcohol) was measured using a sterile medical syringe and placed on a room temperature glass plate.
2) The glass plate was gently heated to 95 F using an electric hotplate with even heat distribution to minimize the chance of higher temperatures on various parts of the surface. Temperature was monitored by infrared thermometer.
3) The dehydration process was closely monitored by gently tilting the plate side to side to evaluate the presence of liquid and the odor was evaluated for the presence of evaporating benzyl alcohol detected by its characteristic aromatic nature.
4) The dehydration process took less than 15 min, at which time the remaining substance on the surface was no longer subjected to movement when the plate was tilted and the aromatic benzyl alcohol vapors were no longer present.
5) The remaining residue was shiny in appearance and mildly sticky when touched.
6) The residue was then removed using a clean razorblade. The residue then took on the appearance of a very slightly off-white, slightly sticky crystalline substance.
7) The substance was then transferred to clean wax paper.
8) The wax paper was used to transfer the substrate to a test-tube containing 9.75 ml of room temperature distilled water and was mildly agitated to increase the rate of dissolution.
9) 0.25 ml of a 20% lipid emulsion was then added to the solution from step 8.
10) For the pediatric/canine dosage, the process was the same save for the following differences:
   a) 5 ml of FDF doxapram was placed on the room temperature plate, instead of 15 ml.
   b) The resultant substance from (a) was added to 3.25 ml of distilled water.
   c) 83 μl of a 20% lipid emulsion was then added to the solution from step (b) using a micropipette.

### Example 3—Effect of Lipid on Doxapram Rate of Absorption in Canines after Intramuscular Injection

The administration of doxapram to increase respiratory drive via intramuscular injection enables easier administration versus intravenous administration, but in turn hinders doxapram absorption. To address this issue, the encapsulation of doxapram in lipid emulsion (from Example 2) was explored to increase the rate of doxapram absorption and minimize time from intramuscular injection to time of action.

### Method

1) Three beagles were sedated with an intramuscular injection of dexmedetomidine.
2) An IV was placed in the cephalic vein.
3) General anesthesia was induced using propofol, and the beagles were intubated.
4) Anesthesia was maintained using isoflurane and the following vitals were monitored: heart rate, blood pressure, respiratory rate, O₂ saturation and core temperature.
5) After a 15 minute stabilization period, 1 ml of blood was drawn to establish a baseline (zero) doxapram blood level.
6) Doxapram (3 mg/kg) was injected intramuscularly.
7) At five minute intervals, additional 1 ml blood draws were performed for 60 minutes after the doxapram injection (12 blood draws total).
8) After the final blood draw, the canines were allowed to emerge from anesthesia, monitored, then returned to their respective kennels.
9) The animals were allowed one week for the doxapram levels to return to zero before beginning the next arm of the study.
10) In the second arm of the study, steps 1-9 were repeated, except the doxapram injection included the addition of a 20% lipid emulsion so that the final concentration of lipid was 0.5%.
11) In the third arm of the study, steps 1-9 were repeated, except the doxapram injection included the addition of a 20% lipid emulsion so that the final concentration of lipid was 1.5%.
12) The collected blood samples were sent to a laboratory to analyze the concentrations of doxapram.

Table 1 provides a summary of the three study arms.

TABLE 1
Exemplary study arms

| | Doxapram | Lipid Emulsion |
|---|---|---|
| Study Arm | (mg/kg) | % Final |
| First | 3 | 0 |
| Second | 3 | 0.5 |
| Third | 3 | 1.5 |

### Results

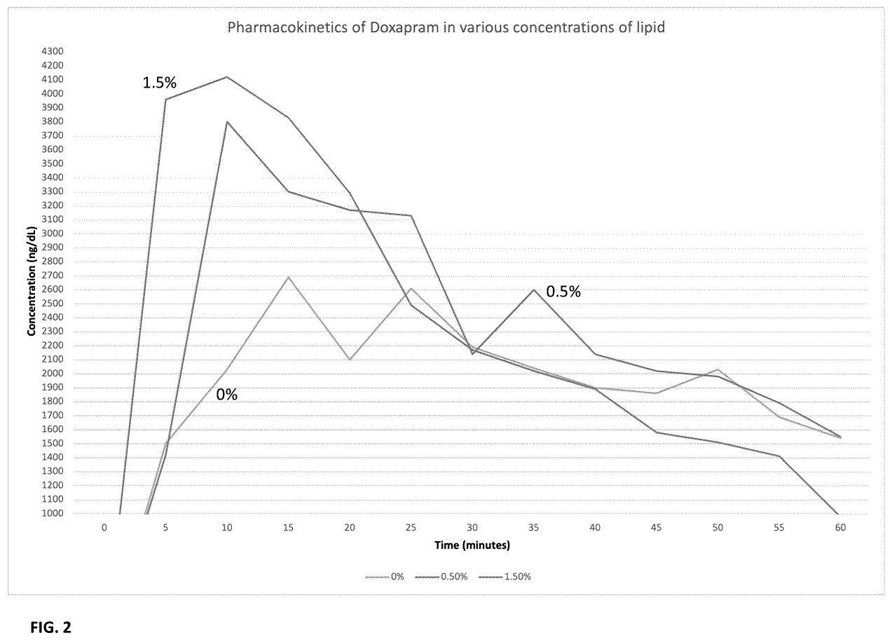

Analysis of the blood samples demonstrated a lipid emulsion concentration dependent rate of uptake increase of doxapram, i.e., doxapram was absorbed more quickly when formulated with 1.5% lipid emulsion, compared to 0.5% lipid emulsion. Both formulations containing lipids were absorbed more quickly than the standard, commercially available, doxapram formulation (i.e., 0% lipid emulsion). Results for all three study arms are shown in FIG. 2.

### CONCLUSION

Doxapram in lipid emulsion are absorbed more quickly after intramuscular injection than in the form without the lipid emulsion. These results support the addition of a lipid emulsion to c doxapram to enable effective intramuscular administration.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-21. (canceled)

22. A method for stimulating respiration, comprising administering a pharmaceutical composition to a subject in need thereof, wherein the pharmaceutical composition comprises:
i) a unit dose of doxapram from about 5 mg to about 600 mg; and
ii) a lipid emulsion, wherein the lipid emulsion ranges from about 0.1% to about 10% by weight of the pharmaceutical composition, and wherein the lipid emulsion comprises soybean oil and egg phospholipid.

23. The method of claim 22, wherein the lipid emulsion comprises glycerin.

24. The method of claim 22, wherein the concentration of doxapram is at least about 20 mg/mL in the pharmaceutical composition.

25. The method of claim 22, wherein the lipid emulsion ranges from about 0.5% to about 1.5% by weight of the pharmaceutical composition.

26. The method of claim 25, wherein the subject has drug-induced post-anesthesia respiratory depression, apnea, respiratory or central nervous system depression due to drug overdose, acute respiratory insufficiency superimposed on chronic obstructive pulmonary disease (COPD), or cardiopulmonary arrest.

27. A method for stimulating respiration, comprising administering a first pharmaceutical composition and a second pharmaceutical composition to a subject in need thereof,
wherein the first pharmaceutical composition comprises a unit dose of naloxone of about 1 mg to about 3 mg and a unit dose of doxapram of about 50 mg to about 150 mg, and the second pharmaceutical composition comprises a unit dose of doxapram of about 100 mg to about 300 mg and a lipid emulsion, and
wherein the lipid emulsion is about 0.1% to about 10% by weight of the second pharmaceutical composition, and wherein the lipid emulsion comprises soybean oil and egg phospholipid.

28. The method of claim 27, wherein the subject has respiratory or central nervous system depression due to drug overdose.

29. The method of claim 28, wherein the first pharmaceutical composition comprises a unit dose of naloxone of about 0.01 mg/kg body weight to about 1 mg/kg body weight and a unit dose of doxapram of about 0.1 mg/kg body weight to about 10 mg/kg body weight and the second pharmaceutical composition comprises a unit dose of doxapram of about 0.2 mg/kg body weight to about 20 mg/kg body weight.

30. The method of claim 29, wherein the doxapram reaches a maximum plasma concentration (Cmax) in about 15 minutes to about 30 minutes and maintains a plasma concentration of more than about 5% of said Cmax for at least once hour, after a single dose of said pharmaceutical composition by intramuscular injection.

31. The method of claim 22, wherein the pharmaceutical composition is administered intramuscularly.

32. The method of claim 22, wherein the lipid emulsion ranges from about 0.5% to about 1.5% by weight of the pharmaceutical composition, and wherein the pharmaceutical composition is administered intramuscularly.

33. The method of claim 22, wherein the concentration of doxapram is about 1 mg/mL to about 20 mg/mL in the pharmaceutical composition.

34. A method for stimulating respiration, comprising administering a pharmaceutical composition to a subject in need thereof, wherein the pharmaceutical composition comprises:
i) a unit dose of doxapram from about 5 mg to about 600 mg; and
ii) a lipid emulsion, wherein the lipid emulsion ranges from about 0.5% to about 1.5% by weight of the pharmaceutical composition, and wherein the lipid emulsion comprises soybean oil, egg phospholipid, and glycerin,
wherein the pharmaceutical composition is administered intramuscularly.

35. The method of claim 34, wherein the lipid emulsion is about 1.5% by weight of the pharmaceutical composition.
